(19) United States
(12) Patent Application Publication
(10) Pub. No.: US 2023/0037975 A1
(43) Pub. Date: Feb. 9, 2023

(54) USE OF CANNABINOID COMPOUNDS IN ORAL CARE COMPOSITIONS TO INHIBIT THE GROWTH OF P. GINGIVALIS

(71) Applicant: Demetrix, Inc., Berkeley, CA (US)

(72) Inventors: Cynthia W. Bryant, Raleigh, NC (US); Alison Watta, Durham, NC (US)

(21) Appl. No.: 17/877,907

(22) Filed: Jul. 30, 2022

Related U.S. Application Data
(60) Provisional application No. 63/227,768, filed on Jul. 30, 2021.

(51) Int. Cl. A61K 8/34; A61Q 11/00; A61K 8/368

(52) U.S. Cl. CPC A61K 8/347; A61Q 11/00; A61K 8/368

(57) ABSTRACT

Methods of inhibiting the growth of Porphyromonas gingivalis (“P. gingivalis”) in the oral cavity using cannabinoid compounds are described herein. The cannabinoid compounds can include one or more of cannabinol cannabigerol (“CBG”), cannabigerolic acid (“CBGA”), cannabidiolic acid (“CBDA”), and (+)-cannabidiol (“((+)-CBD”), cannabichromene (“CBC”), cannabinol (“CBN”), cannabidivarin (“CBDV”), cannabidol (“CBD”), cannabidol-C₄ (“CBD-C₄”), cannabigerivarin (“CBGV”), cannabigerol butyl (“CBG-C4”), cannabichromenic acid (“CBCA”), cannabicyclolic acid (“CBLA”), and cannabinolic acid (“CBNA”), cannabidol-C₂ (“CBD-C₂”) and cannabigerolic acid butyl (“CBGA-C4”). The methods can be used to treat or prevent periodontal disease. Compositions and articles including the cannabinoid compounds are further disclosed.

### CROSS-REFERENCE TO RELATED APPLICATIONS

The present application claims the priority benefit of U.S. Provisional Patent App. Ser. No. 63/227,768, filed Jul. 30, 2021, which is hereby incorporated herein by reference.

### TECHNICAL FIELD

The present disclosure generally relates to the use of cannabinoid compounds in oral care compositions to improve oral health. The cannabinoid compounds can inhibit the microbial growth of Porphyromonas gingivalis.

### BACKGROUND

Porphyromonas gingivalis (“P. gingivalis”) is an anaerobic and Gram-negative bacteria that can be found in the human oral cavity. P. gingivalis is associated with periodontal disease and is believed to cause a microbial shift in the oral cavity that allows for uncontrolled growth of commensal microbial species. The presence of P. gingivalis in the oral cavity causes inflammation and eventual deterioration of gum tissue. The development of periodontal disease is further associated with a higher risk of developing cardiovascular disease.

### DETAILED DESCRIPTION

Periodontal disease, colloquially referred to as gum disease, is an oral infection that causes inflamed gums (gingivitis), bad breath, and eventual loss of teeth. Although periodontal disease can be caused by multiple infectious agents, infection with Porphyromonas gingivalis (“P. gingivalis”) is a major etiological agent that significantly increases the likelihood that periodontal disease will develop by modifying the host inflammatory response and by modifying the microbial environment to allow for uncontrolled growth of commensal microbial species. The development of periodontal disease is further associated with a higher risk of developing cardiovascular disease making prevention of periodontal disease even more critical.

Unfortunately, controlling or inhibiting the growth of P. gingivalis in the oral cavity is difficult. As can be appreciated, P. gingivalis is a Gram-negative bacteria and is difficult to control with both existing medicines and oral care products. For example, the current standard of care to treat P. gingivalis is surface debridement followed by adjunctive treatment with antiseptic agents and/or antibiotics.

However, known antiseptic agents and antibiotics have numerous shortcomings. Known antiseptic agents such as chlorhexidine cause discoloration of teeth, alteration in taste, supragingival calculus formation, bitter taste, and other side effects which discourage use. Antibiotics such as tetracyclines, macrolides, lincosamides, β-lactams, and nitroimidazoles have also been used to attempt to inhibit or control P. gingivalis. However, treatments with existing classes of antibiotics have been largely ineffective against P. gingivalis when used topically or systematically. Although research is ongoing to locate more effective antibiotics, there is an unmet need for an agent which can inhibit the growth of P. gingivalis.

As will be described herein, the present disclosure describes the use of one or more cannabinoid compounds to inhibit the growth of P. gingivalis. Reduction, or elimination, of P. gingivalis in the oral cavity will significantly decrease the likelihood that periodontal disease will develop and can also be used to treat existing periodontal disease. These cannabinoid compounds can be included in a variety of different oral care products.

It has specifically been found that cannabigerol (“CBG”), cannabigerolic acid (“CBGA”), cannabidiolic acid (“CBDA”), and (+)-cannabidiol (“((+)-CBD”) each have a minimum inhibitory concentration (“MIC”) against P. gingivalis of less 1 μg/mL. Such a MIC value means that these cannabinoid compounds are highly effective at inhibiting the growth of P. gingivalis. Thus, these cannabinoid compounds can be included in various oral care products to reduce, or eliminate, an infection caused by P. gingivalis to prevent periodontal disease.

Other cannabinoid compounds were also found to be effective against P. gingivalis, but at higher concentrations. For example, cannabichromene (“CBC”) exhibited a MIC of 1.56 μg/mL against P. gingivalis while cannabinol (“CBN”), cannabidivarin (“CBDV”), cannabidol (“CBD”), cannabidol-C₄ (“CBD-C₄”), cannabigerivarin (“CBGV”), cannabigerol butyl (“CBG-C4”), cannabichromenic acid (“CBCA”), cannabicyclolic acid (“CBLA”), and cannabinolic acid (“CBNA”) each exhibited a MIC of 3.125 μg/mL against P. gingivalis. Cannabidol-C₂ (“CBD-C₂”) and cannabigerolic acid butyl (“CBGA-C4”) each exhibited a MIC of 6.25 μg/mL against P. gingivalis. These additional cannabinoid compounds can, in certain embodiments, also be used independently, or as an additional active, against P. gingivalis in the oral care products described herein.

Other cannabinoid compounds were not found to be effective at inhibiting the growth of P. gingivalis when present at low concentrations. For example, cannabidivarinic acid (“CBDVA”) and cannabigerovarinic acid (“CBGVA”) were found to have a MIC of 25 μg/mL against P. gingivalis. Cannabidiorcol (“CBD-C1”) and cannabicyclol (“CBL”) were found to have a MIC of 100 μg/mL and greater than 200 μg/mL respectively against P. gingivalis. As can be appreciated however, although less effective, cannabinoid compounds with a MIC of less than 200 μg/mL can be used to inhibit the growth of P. gingivalis in certain embodiments.

Prior to the present discovery, it was not appreciated which specific cannabinoid compounds could act to inhibit the growth of P. gingivalis. By selecting the cannabinoid compounds with the greatest effectiveness against P. gingivalis, the present disclosure provides improved methods of inhibiting the growth of P. gingivalis, and methods of preventing, or treating, periodontal disease.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to treat gastric acid diseases with specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally derived cannabinoid compounds offer benefits in regard to the treatment of periodontal disease by inhibiting the growth of P. gingivalis not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of treating P. gingivalis that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally derived cannabinoid compounds are sufficiently pure and/or if any containments are sufficiently well understood.

Generally, oral care products that reduce, or eliminate the growth of P. gingivalis can include an effective concentration of one or more of CBG, CBGA, CBDA, or (+)-CBD. In certain embodiments, the one or more cannabinoid compounds can alternatively, or further, include one or more of CBC, CBDVA, CBN, CBDV, CBD, CBD-C₂, CBD-C₄, CBGV, CBG-C₄, CBCA, CBLA, or CBNA. In certain embodiments, each of the foregoing cannabinoid compounds can be included while in other embodiments, only a subset of such cannabinoid compounds can be included. In certain embodiments, only a single cannabinoid compound can be included.

The effective concentration of the one or more cannabinoid compound can vary depending on factors such as the desired effect of treatment, the severity of the P. gingivalis infection, the severity of the periodontal disease, the duration of treatment, and the method of delivering the cannabinoid compounds to the subject. For example, in certain embodiments, a chewing gum can include a smaller concentration of the cannabinoid compounds because the gum will provide longer exposure of the cannabinoid compounds to the desired areas of the oral cavity than other oral care products such as a mouth wash.

In certain embodiments, the concentration of the cannabinoid compounds can be about equal to the MIC value of the cannabinoid compounds. In other embodiments, the concentration of the cannabinoid compounds can be greater than the MIC value such as in examples where oral contact is shorter in duration (e.g., a toothpaste or mouthwash). In certain embodiments, the cannabinoid compounds can be included in the oral care product at a concentration of about 200 μg per mL, or g, of the oral care product or less.

In certain embodiments, the compositions, products, articles, and methods described herein can be substantially or entirely free of cannabinoid compounds other than of CBG, CBGA, CBDA, (+)-CBD, CBC, CBDVA, CBN, CBDV, CBD, CBD-C₂, CBD-C₄, CBGV, CBG-C₄, CBCA, CBLA, CBNA, CBDVA, CBGVA, and CBD-C1. For example, the compositions, products, articles, and methods can be substantially or entirely free of tetrahydrocannabinol (“THC”). As used herein, substantially free can mean less than about 5%, less than about 4%, less than about 3%, less than about 2%, less than about 1%, less than about 0.5%, less than about 0.1%, or less than about 0.01%. In certain embodiments, the cannabinoid compounds can be produced using non-horticulturally derived methods such as through chemical synthesis (e.g., organic synthesis reactions) or through modification of yeast and/or bacterial cells to produce the cannabinoid compounds in high purity. However, in certain embodiments, cannabinoid compounds can also be a natural product, e.g., an extract of a cannabis plant if sufficiently pure. In certain embodiments, substantially pure means that the isolated cannabinoid compound, when added, includes about 3% or less of contaminants, about 2% or less of contaminants, about 1% or less of contaminants, about 0.5% or less of contaminants, about 0.1% or less of contaminants, or about 0.01% or less of contaminants.

In certain embodiments, the compositions, products, articles, and methods described herein can be utilized on a predetermined schedule (e.g., nightly, twice daily, etc.) or can be utilized on an as-needed basis. In certain embodiments, the predetermined schedule can be based on the half-life of the cannabinoid compounds as well as the release dynamics of the cannabinoid compounds. In certain embodiments, it can be useful to use the compositions, products, articles, and methods described herein as part of routine oral health care to prophylactically treat against infection with P. gingivalis. In other embodiments, the oral health care products described herein can be used only by oral health professionals (e.g., dentists, oral surgeons, etc.) or under their supervision.

Generally, the cannabinoid compounds can be included in any known oral health care product. For example, the cannabinoid compounds can be included in toothpaste, mouthwash, gargles, solutions, drops, emulsions, suspensions, liquids, gels, lozenges, mints, chewable, wipes, powders, or floss. The oral health care products can be intended for use by consumers and patients and/or be intended for use by dental professionals (e.g., dental hygienists, dentists and oral surgeons).

In certain embodiments, the cannabinoid compounds can be incorporated into an oral care composition, product or device, such as a toothpaste, a tooth gel, powder, wipe, a mouthwash or rinse, or a dental floss. In certain embodiments, the cannabinoid compounds can be included in a separate oral care composition, product or device which will be used separately from other compositions, products and devices employed in the prophylactic oral care regimen. For example, the cannabinoid compounds can be incorporated into a mouthwash or rinse, a gum, a lozenge or a chewable tablet.

In certain embodiments, the cannabinoid compounds can be included in dentifrices such as toothpastes, tooth gels, tooth powders and liquid dentifrices. Toothpastes and tooth gels generally include a dental abrasive, a surfactant, a thickening agent, a humectant, a flavoring agent, a sweetening agent, a coloring agent and water. Toothpastes and tooth gels can also include opacifying agents, anti-caries agents, anti-calculus agents, tooth whitening agents, and other optional ingredients. Liquid dentifrices can comprise water, ethanol, a humectant, a surfactant, a thickening agent, an abrasive, an anti-caries agent, a flavoring agent and a sweetening agent.

In certain embodiments, the cannabinoid compounds can be included in gels such as dentifrice gels, non-abrasive gels and subgingival gels. Non-abrasive gels and subgingival gels generally include a thickening agent, a humectant, a flavoring agent, a sweetening agent, a coloring agent, and water. Such gels can also include one or more anti-caries agents and/or anti-calculus agents.

In certain embodiments, the cannabinoid compounds can be included in creams. The creams can include a thickening agent, a humectant and a surfactant, and can include a flavoring agent, a sweetening agent, and a coloring agent.

In certain embodiments, ointments suitable for oral use can generally include one or more of the following: fats, oils, waxes, paraffins, silicones, plastibase, alcohols, water, humectants, surfactants, thickening agents, talc, bentonites, zinc oxide, aluminum compounds, preservatives, antiviral compounds, and other ingredients.

In certain embodiments, mouthwashes, rinses, gargles and sprays can include water, ethanol, and/or a humectant, and can also include a surfactant, a flavoring agent, a sweetening agent, and a coloring agent, and can include a thickening agent and one or more anti-caries agents and/or anti-calculus agents.

Suitable oral care devices include materials (such as sutures and sponges), flosses, tapes, chips, strips, fibers, a toothpick or rubber tip, syringes, dental implants and dental appliances (such as trays and troughs that fit over and cover the teeth and, optionally, the periodontal tissue) having one or more cannabinoid compounds adhered to, absorbed into, bound to, attached to, entrapped in, enclosed in, coated onto, or otherwise incorporated into, them.

In certain embodiments, a compressed chewable tablet comprises a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including the cannabinoid compounds, can be dry blended and compressed into a tablet.

In certain embodiments, the cannabinoid compounds described herein can be included in a tooth whitening composition which is applied to the teeth by means of a dental tray or trough. Alternatively, a separate composition comprising the cannabinoid compounds can be applied to the teeth in a cleaned or different tray or trough. In additional or alternative embodiments, a wash or rinse comprising the cannabinoid compounds could be used to rinse the mouth before and/or after the application of the tooth whitening composition or toothpaste. In certain embodiments, the cannabinoid compounds can be applied using a flexible strip

As can be appreciated, chewing gum compositions generally include a gum base, a flavoring agent and a sweetening agent. Suitable gum bases include jelutong, rubber, latex, chicle, and vinylite resins, desirably with conventional plasticizers or softeners. Examples of suitable plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. The cannabinoid compounds can be incorporated into a gum base by, e.g., stirring them into a warm gum base or coating them onto the outer surface of the gum base.

In certain embodiments, additional ingredients can be added to the oral care compositions, products and articles described herein. Conventional ingredients used in oral care compositions include water, alcohols, humectants, surfactants, thickening agents, abrasives, flavoring agents, sweetening agents, antimicrobial agents, anti-caries agents, anti-plaque agents, anti-calculus agents, and pH-adjusting agents.

Humectants suitable for use in oral care compositions include edible polyhydric alcohols such as glycerol, sorbitol, xylitol, butylene glycol, polyethylene glycol, propylene glycol, mannitol, and lactitol. Humectants help keep oral care compositions, such as pastes, from hardening upon exposure to air, give oral care compositions a moist feel to the mouth, and may impart desirable sweetness.

Surfactants include anionic, nonionic, amphoteric, zwitterionic and cationic synthetic detergents. Anionic surfactants include the water-soluble salts of alkyl sulfates having 8-20 carbon atoms in the alkyl radical (such as sodium alkyl sulfate), the water-soluble salts of sulfonated monoglycerides of fatty acids having from 8-20 carbon atoms (such as sodium lauryl sulfate and sodium coconut monoglyceride sulfonates), sarcosinates (such as sodium and potassium salts of lauroyl sarcosinate, myristoyl sarcosinate, palmitoyl sarcosinate, stearoyl sarcosinate and oleoyl sarcosinate), taurates, higher alkyl sulfoacettes (such as sodium lauryl sulfoacetate), isethionates (such as sodium lauroyl isethionate), sodium laureth carboxylate, sodium dodecyl benezesulfonate, and mixtures of the foregoing. Preferred are the sarcosinates since they inhibit acid formation in the mouth due to carbohydrate breakdown. Nonionic surfactants include poloxamers (sold under the tradename Pluronic), polyoxyethylene sorbitan esters (sold under the tradename Tween), fatty alcohol ethoxylates, polyethylene oxide condensates of alkyl phenols, products derived from the condensation of ethylene oxide with fatty acids, fatty alcohols, fatty amides, polyhydric alcohols, and polypropyleneoxide, ethylene oxide condensates of aliphatic alcohols, long-chain tertiary amine oxides, long-chain tertiary phospine oxides, long-chain dialkyl sulfoxides, and mixtures of such materials. Amphoteric surfactants include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials. Zwitterionic surfactants include derivatives of aliphatic quaternary ammonium, phosphonium and sulfonium compounds in which the aliphatic radical can be a straight or branched chain and wherein one of the aliphatic substituents contains about 8-18 carbon atoms and one contains an anionic water-solubilizing group (such as carboxy, sulfonate, sulfate, phosphate or phosphonate). Cationic surfactants include aliphatic quaternary ammonium compounds having one long alkyl chain containing about 8-18 carbon atoms (such as lauryl trimethylammonium chloride, cetyltrimethylammonium bromide, diisobuytylphenoxyethyldimethylbenzylammonium chloride, coconut alkyltrimetylammonium nitrite, cetylpyridinium fluoride). Certain cationic surfactants can also act as antimicrobials.

Thickening agents include carboxyvinyl polymers, polyvinylpyrrolidone, polyacrylates, carrageenan, cellulose derivatives (e.g., hydroxypropyl cellulose, hydroxypropyl methyl cellulose, methyl cellulose, and hydroxyethyl cellulose), laponite, water-soluble salts of cellulose ethers (such as sodium carboxymethylcellulose and sodium carboxymethyl hydroxyethyl cellulose), natural gums (such as gum karaya, xanthan gum, gum arabic and gum tragacanth), polymeric polyether compounds (such as polyethylene oxide and polypropylene oxide), homopolymers of acrylic acid crosslinked with an alkyl ether of pentaerythritol, alkyl ether of sucrose, carbomers (sold under the tradename Carbopol®, starch, copolymers of lactide and glycolide monomers (the copolymer having an average molecular weight of about 1,000-120,000), colloidal magnesium aluminum silicate and finely divided silica. Thickening agents will be added in amounts sufficient to give a desired consistency to an oral care composition.

Abrasives include silicas (including gels and precipitates), aluminas, calcium carbonates, calcium phosphates, dicalcium phosphates, tricalcium phosphates, hydroxyapatites, calcium pyrophosphates, trimetaphosphates, insoluble polymetaphopsphates (such as insoluble sodium polymetaphosphate and calcium polymetaphosphate), magnesium carbonates, magnesium oxides, resinous abrasive materials (such as particulate condensation products of urea and formaldehyde), particulate thermosetting polymerized resins (suitable resins include melamines, phenolics, ureas, melamine-ureas, melamine-formaldehydes, urea-formaldehydes, melamine-urea-formaldehydes, cross-linked epoxides and cross-linked polyesters), and combinations of the foregoing. Silica abrasives are preferred because they provide excellent dental cleaning and polishing performance without unduly abrading tooth enamel or dentine.

Flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, ethyl butyrate, benzyl acetate, amyl acetate, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing. As can be appreciated, flavoring agents can be used to create the taste of bubblegum, fruits such as strawberry, cherry, grape, apple, etc., and other known artificial and natural flavors.

Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing.

In addition to the flavoring and sweetening agents, the oral care compositions can include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol.

Anti-caries agents include sodium fluoride, stannous fluoride, potassium fluoride, amine fluorides, indium fluoride, sodium monofluorophosphate, calcium lactate, calcium glycerophosphates, strontium salts, and strontium polyacrylates.

Anti-calculus agents include pyrophosphate salts such as dialkali metal pyrophosphate salts and tetraalkali metal pyrophosphate salts (e.g., disodium dihydrogen pyrophosphate, tetrasodium pyrophosphate and tetrapotassium pyrophosphate, in their hydrated and unhydrated forms). Other anti-calculus agents which can be used instead of, or in addition to, the pyrophosphate salts include synthetic anionic polymers (such as polyacrylates and copolymers of maleic anhydride or acid and methyl vinyl ether), polyaminopropane sulfonic acid, zinc citrate trihydrate, polyphosphates (such as tripolyphosphate and hexametaphosphate), polyphosphonates (such as disodium ethane-1-hydroxy-1,1-diphosphonate (EHDP), methanedisphosphonic acid, and 2-phosphonobutane-1,2,4-tricarboxylic acid), and polypeptides (such as polyaspartic acid and polyglutamic acid).

A pH-adjusting agent and/or a buffering agent or agents may need to be included in the oral care compositions. The pH-adjusting agent may be any compound or mixture of compounds that will achieve the desired pH. Suitable pH-adjusting agents include organic and inorganic acids and bases, such as benzoic acid, citric acid, potassium hydroxide, and sodium hydroxide. Buffering agents include acetate salts, borate salts, carbonate salts, bicarbonate salts (e.g., an alkali metal bicarbonate, such as sodium bicarbonate (also known as baking soda)), gluconates, tartrates, sulfates, citrates (such as sodium citrate), benzoate salts, nitrate salts (such as sodium and potassium nitrate), phosphate salts (such as potassium and sodium phosphate), and combinations of the foregoing as needed to achieve and maintain the desired pH.

Suitable antioxidants include superoxide dismutase, catalase, glutathione peroxidase, ebselen, glutathione, cysteine, N-acetyl cysteine, penicillamine, allopurinol, oxypurinol, ascorbic acid, α-tocopherol, Trolox (water-soluble α-tocopherol), vitamin A, β-carotene, fatty-acid binding protein, fenozan, probucol, cyanidanol-3, dimercaptopropanol, indapamide, emoxipine, dimethyl sulfoxide, and others.

Methods of contacting tissues of the mouth with oral care compositions are well known in the art. Suitable methods include rinsing the tissue with a solution (e.g., a mouthwash, rinse, spray, liquid dentifrice, or other solution), brushing the teeth with a dentifrice (e.g., a toothpaste, tooth gel, or powder), applying a non-abrasive solution, gel, paste, cream or ointment directly to the tissue (with or without the use of an applicator) (including fluoride varnishes), chewing gum, chewing or sucking a lozenge, mint or tablet, and many other means of topical application. Suitable applicators for applying oral care compositions, such as solutions, gels, pastes, creams and ointments, to a tissue include a swab, a stick, a plastic paddle, a dropper, a syringe, a strip, a finger, or a dental tray or appliance which allows for immersion of the teeth and, optionally, the periodontal tissue in, e.g., a gel or solution thereof. In certain embodiments, oral care compositions further comprise, for instance, sutures can be used to close a surgical wound or a wound resulting from a tooth extraction, dental floss can be used to floss the teeth, etc. The treatment of the tissue can be prophylactic treatment. For instance, the tissue may be treated as part of a prophylactic oral care regimen.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising one or more cannabinoid as discussed herein, the wound(s) caused by the surgery or tooth extraction could be closed with sutures having one or more cannabinoid as discussed herein incorporated into them, and/or the mouth could be rinsed immediately after the surgery or tooth extraction, and/or at intervals thereafter, with a solution comprising one or more cannabinoid as discussed herein.

### Examples

To determine the ability of the cannabinoid compounds described herein to reduce, or eliminate, P. gingivalis a minimum inhibitory concentration (“MIC”) assay was performed. The assay was performed by Charles River Laboratories International (Wilmington, Mass.) in accordance with industry standards. The results are depicted in Table 1.

TABLE 1

| Cannabinoid | MIC (μg/mL) |
|---|---|
| CBG | 0.78 |
| CBGA | <=0.097 |
| CBC | 1.56 |
| CBDVA | 25 |
| CBL | >200 |
| CBN | 3.125 |
| CBDV | 3.125 |
| CBDA | 0.39 |
| CBD | 3.125 |
| CBD-C1 | 100 |
| CBD-C2 | 6.25 |
| CBD-C4 | 3.125 |
| (+)-CBD | 0.78 |
| CBGV | 3.125 |
| CBGVA | 25 |
| CBG-C4 | 3.125 |
| CBGA-C4 | 6.25 |
| CBCA | 3.125 |
| CBLA | 3.125 |
| CBNA | 3.125 |

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

What is claimed is:

1. An oral care composition comprising one or more cannabinoid compounds, the oral care composition inhibiting the growth of Porphyromonas gingivalis (“P. gingivalis”).

2. The oral care composition of claim 1, wherein the one or more cannabinoid compounds comprise cannabigerol (“CBG”), cannabigerolic acid (“CBGA”), cannabidiolic acid (“CBDA”), and (+)-cannabidiol (“((+)-CBD”), cannabichromene (“CBC”), cannabinol (“CBN”), cannabidivarin (“CBDV”), cannabidol (“CBD”), cannabidol-C₄ (“CBD-C4”), cannabigerivarin (“CBGV”), cannabigerol butyl (“CBG-C4”), cannabichromenic acid (“CBCA”), cannabicyclolic acid (“CBLA”), and cannabinolic acid (“CBNA”), cannabidol-C₂ (“CBD-C₂”) cannabigerolic acid butyl (“CBGA-C4”), cannabidivarinic acid (“CBDVA”), cannabigerovarinic acid (“CBGVA”), and cannabidiorcol (“CBD-C1”).

3. The oral care composition of claim 1, wherein the one or more cannabinoid compounds have a minimum inhibitory concentration (“MIC”) against P. gingivalis of less than about 200 μg per mL.

4. The oral care composition of claim 1, wherein the one or more cannabinoid compounds have a MIC against P. gingivalis of about 100 μg per mL or less.

5. The oral care composition of claim 1, wherein the one or more cannabinoid compounds have a MIC against P. gingivalis of about 50 μg per mL or less.

6. The oral care composition of claim 1, wherein the one or more cannabinoid compounds have a MIC against P. gingivalis of about 25 μg per mL or less.

7. The oral care composition of claim 1, wherein the one or more cannabinoid compounds have a MIC against P. gingivalis of about 10 μg per mL or less.

8. The oral care composition of claim 1, wherein the one or more cannabinoid compounds have a MIC against P. gingivalis of about 6.25 μg per mL or less.

9. The oral care composition of claim 1, wherein the one or more cannabinoid compounds have a MIC against P. gingivalis of about 3.125 μg per mL or less.

10. The oral care composition of claim 1, wherein the one or more cannabinoid compounds have a MIC against P. gingivalis of about 1 μg per mL or less.

11. The oral care composition of claim 1 is a liquid or gel.

12. The oral care composition of claim 1 is a toothpaste or mouthwash.

13. The oral care composition of claim 1 is effective in treating periodontal disease.

14. An oral care product comprising the oral health composition of claim 1.

15. The oral care product of claim 14 is a gum or floss.

16. A method of treating or preventing periodontal disease, the method comprising administering a therapeutically effective amount of one or more cannabinoid compounds to the oral cavity; and
wherein the one or more cannabinoid compounds comprise cannabigerol (“CBG”), cannabigerolic acid (“CBGA”), cannabidiolic acid (“CBDA”), and (+)-cannabidiol (“((+)-CBD”), cannabichromene (“CBC”), cannabinol (“CBN”), cannabidivarin (“CBDV”), cannabidol (“CBD”), cannabidol-C₄ (“CBD-C₄”), cannabigerivarin (“CBGV”), cannabigerol butyl (“CBG-C4”), cannabichromenic acid (“CBCA”), cannabicyclolic acid (“CBLA”), and cannabinolic acid (“CBNA”), cannabidol-C₂ (“CBD-C₂”), cannabigerolic acid butyl (“CBGA-C4”), cannabidivarinic acid (“CBDVA”), cannabigerovarinic acid (“CBGVA”), and cannabidiorcol (“CBD-C1”).

17. The method of claim 16, wherein the one or more cannabinoid compounds reduce the amount of Porphyromonas gingivalis (“P. gingivalis”) in the oral cavity.

18. The method of claim 16, wherein the one or more cannabinoid compounds substantially eliminate P. gingivalis in the oral cavity.

19. The method of claim 16 wherein the one or more cannabinoid compounds are applied to the oral cavity using an oral care composition.

20. The method of claim 16, wherein the one or more cannabinoid compounds are applied to the oral cavity using an oral care product.
